(19) United States
(12) Patent Application Publication
(10) Pub. No.: US 2025/0387599 A1
(43) Pub. Date: Dec. 25, 2025

(54) Medical Device for Supporting a Catheter During a Vascular Procedure

(71) Applicant: Surgia X, LLC, Temecula, CA (US)

(72) Inventors: Islam Abudayyeh, Redlands, CA (US); Huu Tam Truong, Redlands, CA (US); Amnart Andy Wongworawat, Temecula, CA (US); Esther Yoonah Kim, Temecula, CA (US)

(73) Assignee: Surgia X, LLC, Temecula, CA (US)

(21) Appl. No.: 18/748,110

(22) Filed: Jun. 20, 2024

(51) Int. Cl. A61M 25/02

(52) U.S. Cl. CPC A61M 25/02

(57) ABSTRACT

The present disclosure relates to a medical device for supporting a catheter during a vascular procedure. The medical device comprises a bottom surface in contact with patient's body surface and a top surface supporting the catheter. The top surface forms an inclined plane or a ramp with the top-bottom connection area/line facing an access site or a skin puncture point of the catheter. The top surface may further comprise a groove to stabilize and guide the catheter during the vascular procedure.

### BACKGROUND

When performing femoral arterial or venous access procedures, the most commonly used method is the modified Seldinger technique. The Seldinger technique, introduced around 1950's, is a method developed to introduce guide wires and catheters into the body of patients. Standard procedure of Seldinger technique generally involves introduction of a needle into the desired body location, loading a guide wire into the needle once the desired body fluid is expressed (or aspirated with a syringe), removing the needle, introducing a tube or catheter onto the wire, and then removing the guide wire afterwards. In order to maintain wire control to achieve desired accuracy and reduce peri-procedural complications, such method requires an operator to have extensive training and relatively good physical conditions. Other femoral arterial or venous access for procedures, such as trans-catheter valve implants, left ventricular assist devices and other structural procedures, impose similar requirements for operation. Currently, however, there is no clinically available device to provide a reliable and stable support for access sheath, needle, guide wires, or catheters in the femoral arterial or venous access procedures.

### SUMMARY OF THE INVENTION

In one aspect, the present invention provides a medical device for supporting a catheter during a vascular procedure. The medical device comprises a bottom surface in contact with patient's body surface and a top surface supporting the catheter. One end of the top surface and one end of the bottom surface are connected forming a connection area or connection line. The top surface forms an inclined plane or a ramp with the connection area/line facing an access site or a skin puncture point of the catheter. The top surface optionally comprises a groove to stabilize and guide the catheter during the vascular procedure. The groove, when present, can hold a catheter having a diameter from about 2 mm to about 20 mm.

In a certain embodiment, the angle between the top surface and bottom surface on the left end is α1 and the angle between the top surface and bottom surface on the right end is α2. The angles of α1 and α2 may be independently from about 1 degree to about 90 degree. In a further embodiment, the angles of α1 and α2 are independently selected from about 45 degrees, about 60 degrees, and about 90 degrees. In some embodiments, the angles of α1 and α2 are equal.

In a certain embodiment, the top and bottom surfaces are further connected by a left surface, a right surface, or both. The medical device exhibits tapering on all edges where every two sides meet with a diameter ranging from 1 mm to 5 mm.

In a certain embodiment, the bottom surface is made of a hard material or a soft material. If made of a hard material, the bottom surface does not change its shape when the device is pressed against the patient's body surface. If made of a soft material, the bottom surface changes its shape when the device is pressed against the patient's body surface and is able to conform to the shape of patient's body surface. The soft material has an apparent hardness/elasticity durometer measurement ranging from 10 to 100 using the Shore 00 index, or from 10 to 90 using the Shore A index, or from 10 to 90 using the Shore D index.

In a certain embodiment, the medical device has a wedge shape. In the wedge shape device, the angles of α1 and α2 are independently set to an angle from 30 to 90 degrees. In a further embodiment, the angles of α1 and α2 in the wedge shape device are equal. In the wedge shape device, the top and bottom surfaces are further connected by a left surface, a right surface, or both. The top surface of the wedge shape device optionally comprise a groove 400. (FIGS. 1B, 2B, and 3B). The groove 400, when present, can hold a catheter having a diameter from about 2 mm to about 20 mm.

In a further embodiment, the bottom surface of the wedge shape device is made of a hard material or a soft material. If made of hard materials, the bottom surface of wedge shape device does not change its shape when the device is pressed against the patient's body surface. If made of a soft material, the bottom surface changes its shape when the device is pressed against the patient's body surface and is able to conform to the shape of patient's body surface. The soft material has an apparent hardness/elasticity durometer measurement ranging from 10 to 100 using the Shore 00 index, or from 10 to 90 using the Shore A index, or from 10 to 90 using the Shore D index. In a certain embodiment, the wedge shape device exhibits tapering on all edges where every two sides meet with a diameter ranging from 1 mm to 5 mm. The wedge shape device further comprises an adhesive allowing the wedge to be secured in position.

In another embodiment, the wedge shape device comprises at least the top, bottom, left and right surfaces, and the interior of the device is hollow allowing a strap or bandage to be passed through it to secure the wedge in position on the body of the patient or bed. In such device, the hollow space has its own top and bottom surfaces and the angle between the top surface and bottom surface of hollow space on the left end is all and the angle between the top surface and bottom surface of the hollow space on the right end is α22. The angles of α11 and α22 can be independently selected from about 15 degrees to about 90 degrees. For example, the angles of α11 and α22 are independently selected from about 45 degrees, about 60 degrees, and about 90 degrees. In a further embodiment, the angles of α11 and α22 are equal. In a preferred embodiment, the angles of α1, α2, α11, and α22 are α11 equal.

### BRIEF DESCRIPTION OF DRAWINGS

FIG. 1A illustrates the left side of the first embodiment of the medical device.

FIG. 1B illustrates a left frontal view of the first embodiment of the device (3D-Illustration of FIG. 1A)

FIG. 2A illustrates the right side of the first embodiment of the wedge shape device

FIG. 2B depicts the right frontal view of the first embodiment of the wedge shape device (3D-Illustration of FIG. 2A)

FIG. 3A depicts the top side of the first embodiment of the wedge shape device.

FIG. 3B illustrates the top view of the first embodiment of the wedge shape device (3D-Illustration of FIG. 3A)

FIG. 4A illustrates the bottom side of the first embodiment of the wedge shape device.

FIG. 4B illustrates the bottom view of the first embodiment of the wedge shape device (3D-Illustration of FIG. 4A).

FIG. 5 illustrates the left frontal view of the first wedge shape device.

FIG. 6 illustrates the right frontal view of the first wedge shape device.

FIG. 7 illustrates the top frontal view of the first wedge shape device.

FIG. 8 illustrates the bottom view of the first wedge shape device.

FIG. 9A illustrates the left side of the third embodiment of the wedge shape device.

FIG. 9B illustrates the left frontal view of the third embodiment of the wedge device (3D-Illustration of FIG. 9A)

FIG. 10A illustrates the right side of the third embodiment of the wedge shape device.

FIG. 10B illustrates the right frontal view of the third embodiment of the wedge device (3D-Illustration of FIG. 10A).

FIG. 11A illustrates the top side of the third embodiment of the wedge device.

FIG. 11B illustrates the top view of the third embodiment of the wedge device (3D-Illustration of FIG. 11A).

FIG. 12A illustrates the bottom side of the third embodiment of the wedge device.

FIG. 12B illustrates the bottom view of the third embodiment of the wedge device (3D-Illustration of FIG. 11A).

FIG. 13 illustrates the left frontal view of the second wedge shape device having a hollow space.

FIG. 14 illustrates the right frontal view of the second wedge shape device having a hollow space.

FIG. 15 depicts the top view of the second wedge shape device having a hollow space.

FIG. 16 depicts the bottom view of the second wedge shape device having a hollow space.

FIG. 17 depicts a side view of an example of the first wedge shape device (the second embodiment), which provides support to access sheath, needle, guide wire, or catheter in the femoral arterial or venous access procedures.

FIG. 18 depicts a top angle view of an example of the first wedge shape device (the second embodiment), which provides support to access sheath, needle, guide wire, or catheter in the femoral arterial or venous access procedures.

FIG. 19 depicts a top angle view of an example of the second wedge shape device (the fourth embodiment), which provides support to access sheath, needle, guide wire, or catheter in the femoral arterial or venous access procedures.

FIG. 20 depicts a typical femoral arterial or venous access procedure.

FIG. 21 depicts the first embodiment showing tapering (round edges), where the two sides meet.

### DETAILED DESCRIPTION OF INVENTION

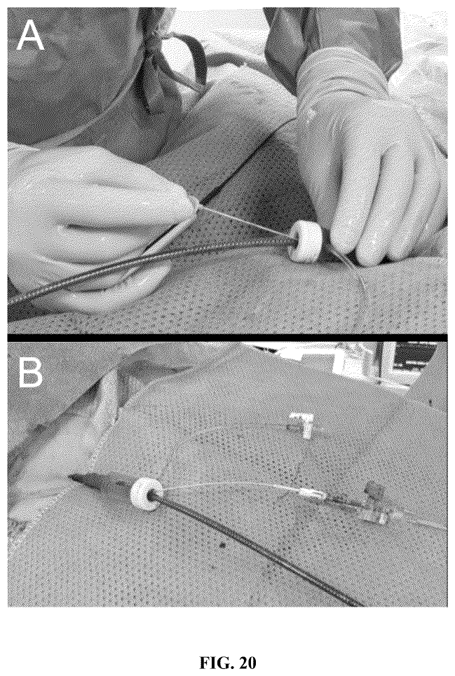

A successful femoral arterial or venous access procedure requires accurate controls of among others, the needle, guide wire, access sheath of a catheter when these components of the catheter are insertion into the blood vessel of a patient in need. Accurate controls of the catheter during the procedure can also reduce peri-procedural complications. Generally speaking, a surgeon, when performing femoral arterial or venous access procedures, has to advance the needle through the skin of a patient in need at an angle anywhere from about 1 to about 90 degrees (See FIG. 20). This requires extensive experience and relatively good physical condition.

Typical femoral arterial or venous access procedures including trans-catheter valve implants, Seldinger technique, left ventricular assist devices and other structural procedures require the operator to anchor the catheter at the skin site of a patient and prevent it from moving so as to avoid vascular injury and stabilize the device (See FIG. 20). Sometimes, it is necessary to secure the devices, such as ventricular support devices or balloon pumps, in the same place or hold the shaft of the device at a constant angle to avoid bleeding and vascular injury for hours if not even days. Currently, such tasks are normally performed by operators using strenuous manual labor.

The present disclosure provides a medical device, which can fulfill the requirements of providing a stable support for femoral arterial or venous access procedures. The disclosed device is also suitable for providing a stable support for the procedure for prolonged period of time.

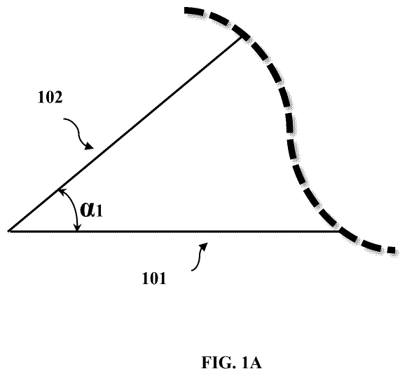

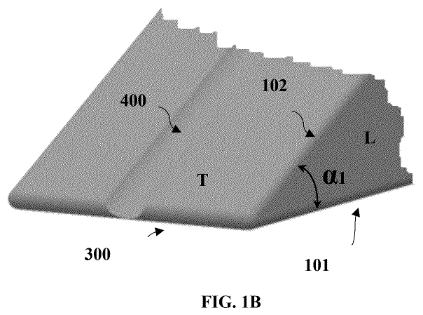

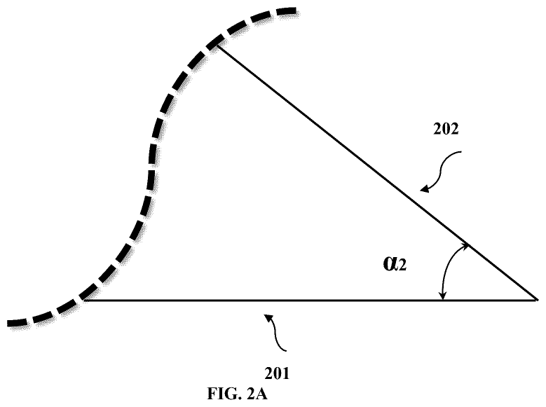

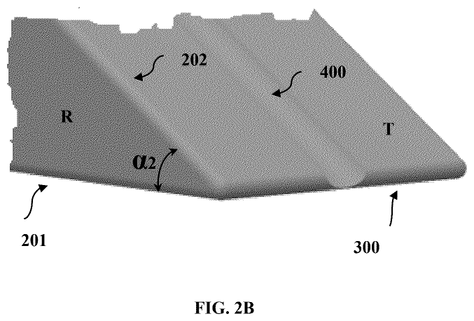

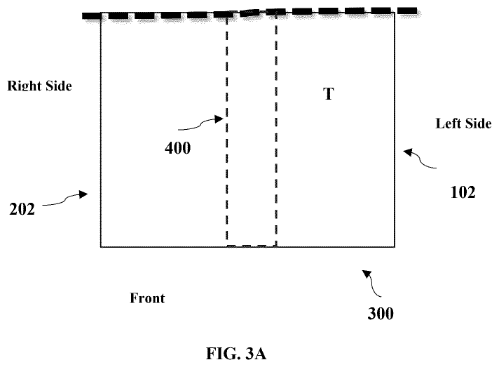

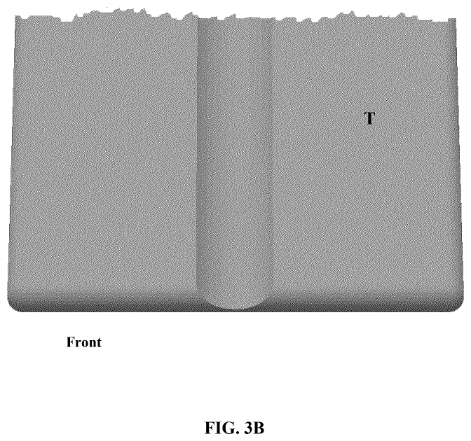

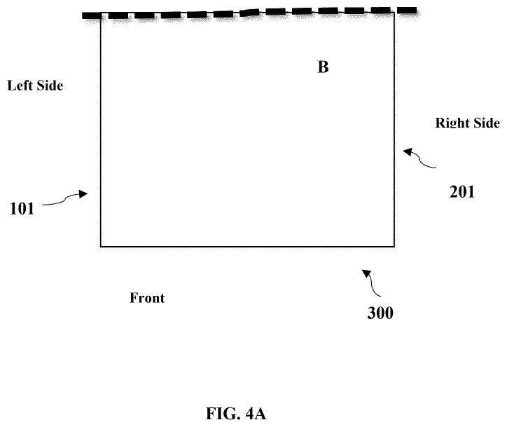

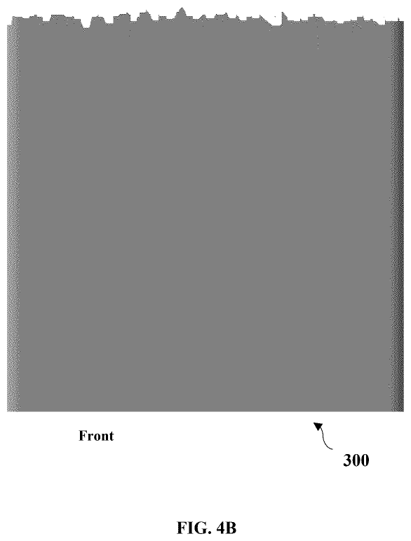

In one aspect, the instant disclosure provides a medical device for supporting an access sheath, a needle, a guide wire, or catheter during a vascular procedure. In a first embodiment, the medical device comprises a bottom surface (B) in contact with patient's body surface, and a top surface (T) supporting the catheter. See FIGS. 1A-4B. In the first embodiment, one end of the top surface and one end of the bottom surface are connected forming a connection area or connection line 300. (FIGS. 1B, 2B, and 2I). The two surfaces may be directly connected without transition or connected with rounded edges.

For example, the rounded edge of 300 exhibits tapering where the two sides meet with a diameter ranging from about 0.10 mm to about 10.00 mm. See edge 300 in FIGS. 1B and 2B. The top surface T forms an inclined plane or a ramp with the connection area/line facing an access site or a skin puncture point of the catheter. The top surface T optionally comprise a groove 400 to stabilize and guide the catheter during the vascular procedure. As shown in FIGS. 1A and 1B, the angle between the top surface T and bottom surface B on the left end is α1. Similarly, the angle between the top surface and bottom surface on the right end is α2. See FIGS. 2A and 2B. Depending on the applications, α1 and α2 may be different or have the same angles.

The medical device of the first embodiment may further comprises a left surface (L) or a right surface (R), or both. (FIGS. 1B and 2B), which, if present, connect the top and bottom surfaces. If the groove 400 is present, it may hold a catheter having a diameter of from about 0.2 mm to about 35 mm. (FIGS. 1B, 2B, 3B, and 17-19).

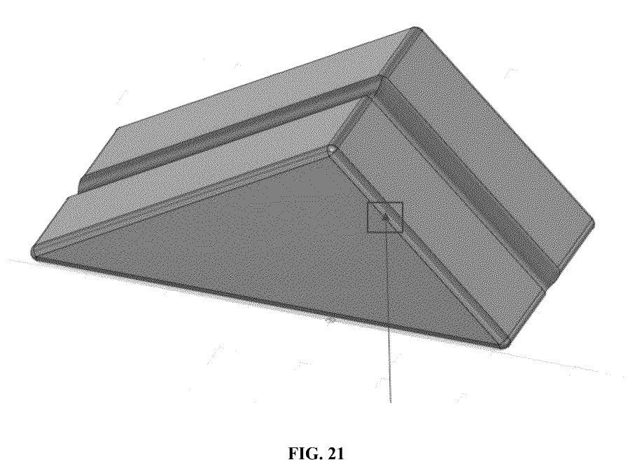

In FIGS. 1A and 1B, 101 is the left edge of bottom surface B and 102 is left edge of the top surface T. In FIGS. 2A and 2B, 201 is the right edge of bottom surface B and 202 is the right edge of top surface T. Similar to edge 300, edges 101, 102, 201, and 202 may be directly formed between two surfaces without transition or connected with rounded edges. If edges 101, 102, 201, and 202 have rounded edges, they exhibit tapering where the two sides meet with a diameter ranging from about 0.10 mm to about 10.00 mm. (See also the highlighted section in FIG. 21).

In the first embodiment, the bottom surface comprises a material, which allows the bottom surface to be pliable when the device is pressed against the patient's body surface. The material has an apparent hardness/elasticity durometer measurement ranging from about 1 to 100 using the Shore 00 index, or from 1 to 180 using the Shore A index, or from 1 to about 100 using the Shore D index. Examples of hardness/elasticity of the bottom face material are in range of from about 1, 5, 10, 15, 20, 25, 30, 35, 40, 45, 50, 55, 60, 65, 70, 75, 80, 85, 90, or 95 to about 100 using the Shore 00 index, in the range of from about 1, 5, 10, 15, 20, 25, 30, 35, 40, 45, 50, 55, 60, 65, 70, 75, 80, or 85, to about 90 using the Shore A index, or in the range of from about 5, 10, 15, 20, 25, 30, 35, 40, 45, 50, 55, 60, 65, 70, 75, 80, or 85, to about 90 using the Shore D index. In a preferred embodiment, the bottom surface material has an apparent hardness/elasticity durometer measurement ranging from about 1 to about 100 using the Shore 00 index, or from about 1 to about 90 using the Shore A index, or from about 1 to about 90 using the Shore D index. In further embodiments, surfaces other than the bottom surface may comprise the same material as described above.

Examples of suitable materials for building the first embodiment include thermoplastic polyurethane (TPU) with suitable elasticity in the ranges as outlined above. The suitable materials can be either among the United States Food and Drug Administration (FDA) certified or non-FDA certified.

Furthermore, in the first embodiment, the bottom surface may comprise an adhesive, which allows the medical device to be secured to the access location such as an acrylic polymer or a similarly applicable adhesive.

In a further embodiment, the diameter of edge 300 may be in the range from about 0.10 mm to about 10.00 mm, from about 0.20 to about 9.50 mm, from about 0.30 to about 9.00 mm, from about 0.30 to about 8.50 mm, from about 0.40 to about 8.00 mm, from about 0.50 to about 7.50 mm, from about 0.60 to about 7.00 mm, from about 0.70 to about 6.50 mm, from about 0.80 to about 6.00 mm, from about 0.90 to about 5.50 mm, from about 1.00 to about 5.00 mm, 1.20 to about 4.80 mm, from about 1.40 to about 4.60 mm, from about 1.60 to about 4.40 mm, from about 1.80 to about 4.60 mm, from about 2.00 to about 4.40 mm, from about 2.20 to about 4.20 mm, from about 2.40 to about 4.00 mm, from about 2.60 to about 3.80 mm, from about 2.80 to about 3.60 mm, or from about 3.00 to about 3.40 mm. In a preferred embodiment, the tapering of edge 300 has a diameter of from about 1.00 mm to about 5 mm. Similarly for edges 101, 102, 201, and 202, when they exhibit tapering, the diameters of these edges fall within the same ranges of edge 300 as disclosed above.

In a further embodiment, the angles of α1 and α2 may be independently in the range of from about 1, 5, 10, 15, 20, 25, 30, 35, 40, 45, 50, 55, 60, 65, 70, 75, 80, or 85 degrees to about 90 degrees. In an even further embodiment, each of the angles of α1 and α2 is independently selected from about 45 degrees to about 90 degrees. In a preferred embodiment, the angles of α1 and α2 are equal.

In a further embodiment, the groove 400 is present. The groove 400 may accommodate a catheter having a diameter of from about 0.2, 0.4, 0.6, 0.8, 0.7, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0 or 34.0 mm to about 35.0 mm. In a preferred embodiment, the groove 400 can hold a catheter having a diameter of from about 2.0 mm to about 20.0 mm.

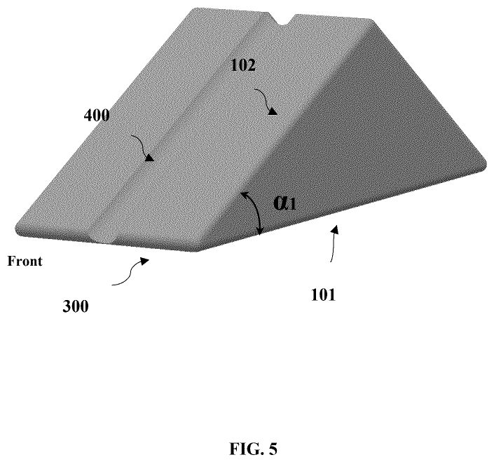

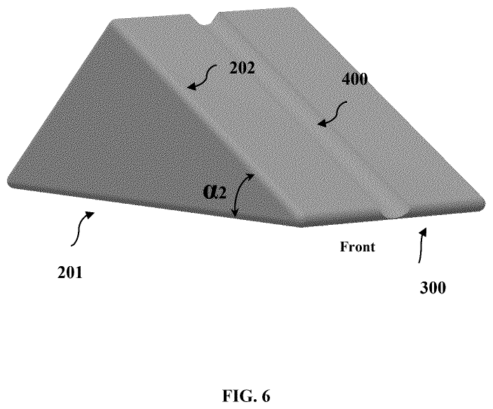

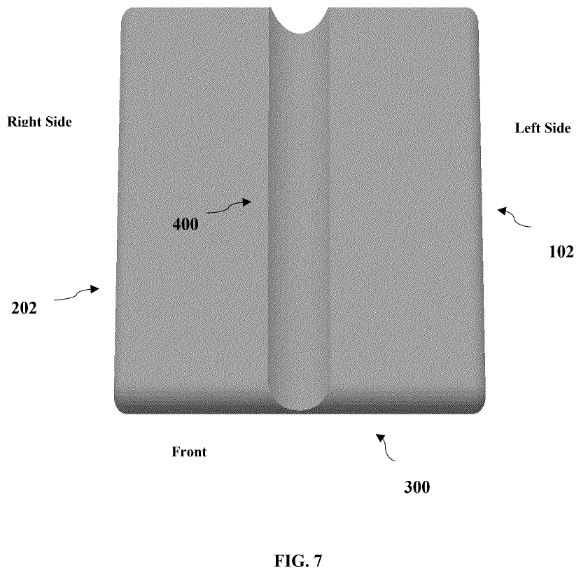

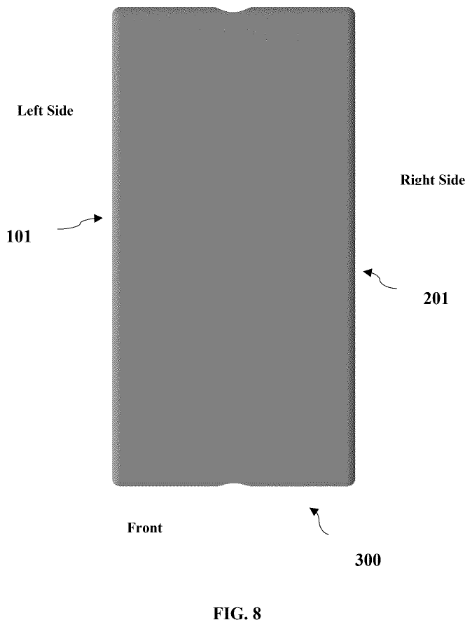

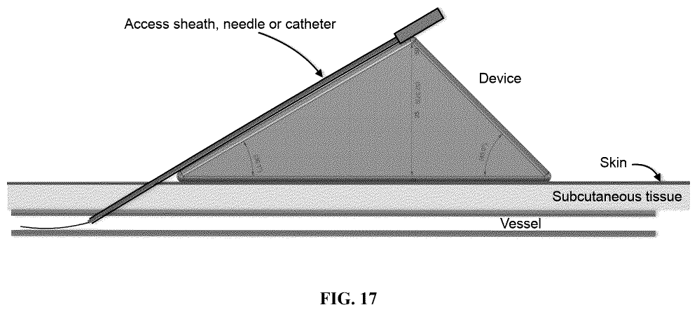

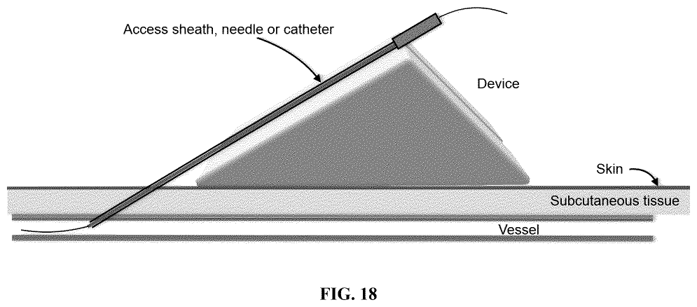

In a second embodiment, the medical device has a wedge shape (a first wedge shape device). (FIGS. 5-8). The first wedge shape device includes and incorporates all the structural components of the first embodiments, i.e., top surface, bottom surface, optional side surfaces, edges 101, 102, 201, 202, 300, and optional groove 400, the angles of α1 and α2, the diameter of catheter the wedge device can hold, the bottom surface material, the bottom surface adhesive, and tapering of the edges. Furthermore, all the structural components of the first wedge shape device, i.e., top surface, bottom surface, optional side surfaces, edges 101, 102, 201, 202, 300, and optional groove 400, the angles of α1 and α2, the diameter of catheter the wedge device can hold, the bottom surface material, the bottom surface adhesive, and tapering of the edges, fall within the same ranges of these structural components of the first embodiments. Furthermore, each of the structural components of the first wedge device and the first embodiment shares the same preferred ranges, respectively. In a more preferred embodiment, the α1 and α2 of the first wedge medical device are equal and have an angle selected from about 45 to about 90 degrees. An example of the application of the first wedge shape device is demonstrated in FIGS. 17-18, where the first wedge shape device provides stable support to access sheath, needle, guide wire, or catheter in the femoral arterial or venous access procedures.

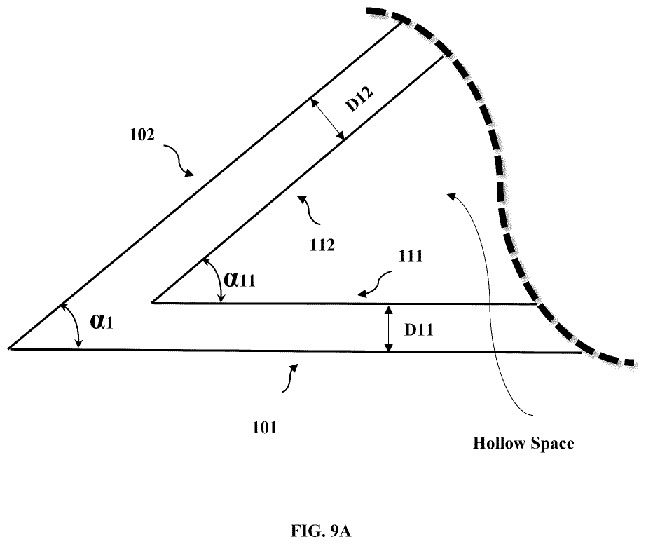

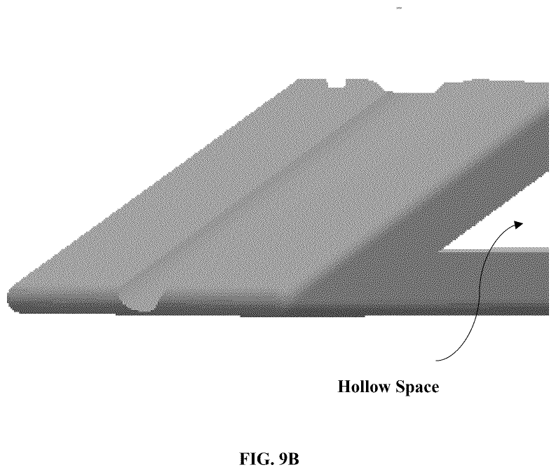

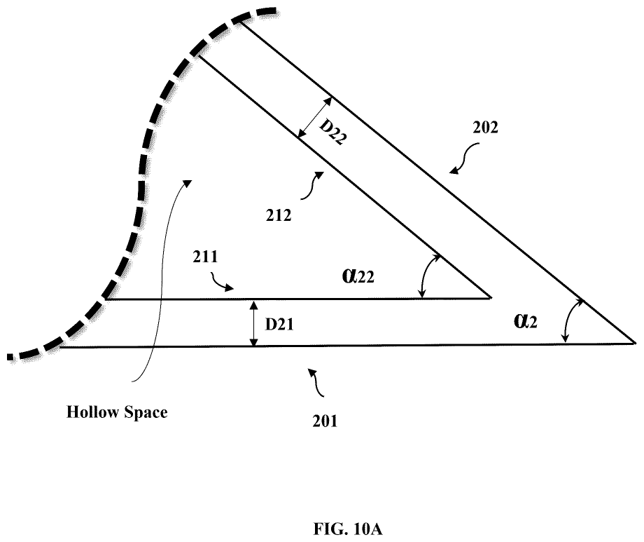

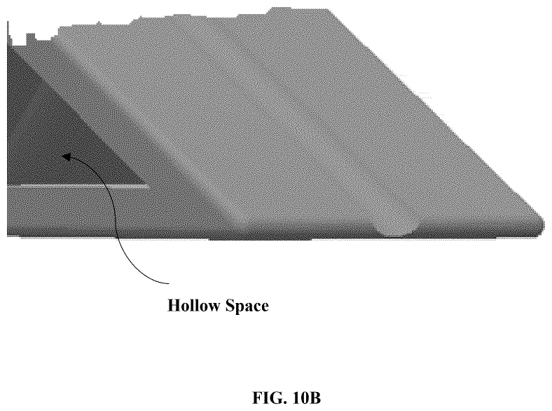

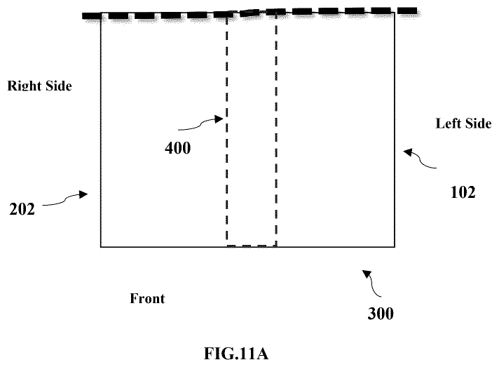

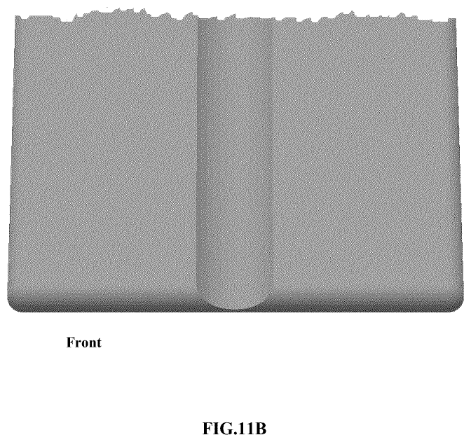

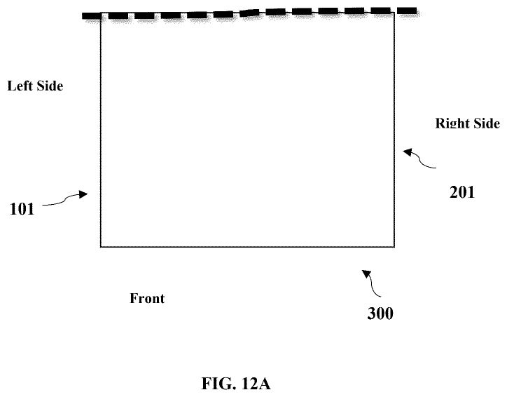

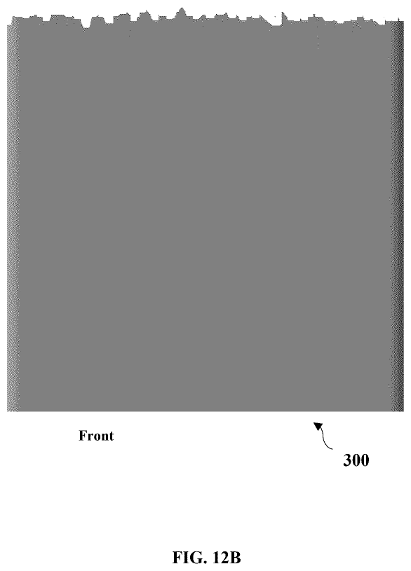

The current disclosure further provides a third embodiment. (FIGS. 9A-12B). The third embodiment comprises all the structural components of the first embodiments, i.e., top surface, bottom surface, side surfaces, edges 101, 102, 201, 202, 300, and optional groove 400, the angles of α1 and α2, the diameter of catheter the wedge device can hold, the bottom surface material, the bottom surface adhesive, and tapering of the edges. The interior of the third embodiment medical device is hollow. (FIGS. 9A-10B). The hollow interior allows straps to pass through to fasten the device to a fixed location of a patient's body surface. Furthermore, the medical device of the third embodiment comprises edges 111 and 112 formed between left surface and hollow interior and edges 211 and 212 formed between the right surface and hollow interior. Each pair of edges 101 and 111, edges 102 and 112, edges 201 and 211, and edges 202 and 212 are independently in parallel to each other (within the pair of edges) or not in parallel to each other (within the pair of edges).

Further in the third embodiment, the angle between edges 111 and 112 is α11. Similarly, the angle between edges 211 and 212 is α22. In a further embodiment, the angles of α11 and α22 may be independently in the range of from about 1, 5, 10, 15, 20, 25, 30, 35, 40, 45, 50, 55, 60, 65, 70, 75, 80, or 85 degrees to about 90 degrees. In an even further embodiment, each of the angles of α11 and α22 is independently selected from about 45 degree to about 90 degree. In a preferred embodiment, the angles of α1 and α11 are equal and the angles of α2 and α22 are equal. In a more preferred embodiment, the angles of α1, α11, α2, and α22 are all equal to each other.

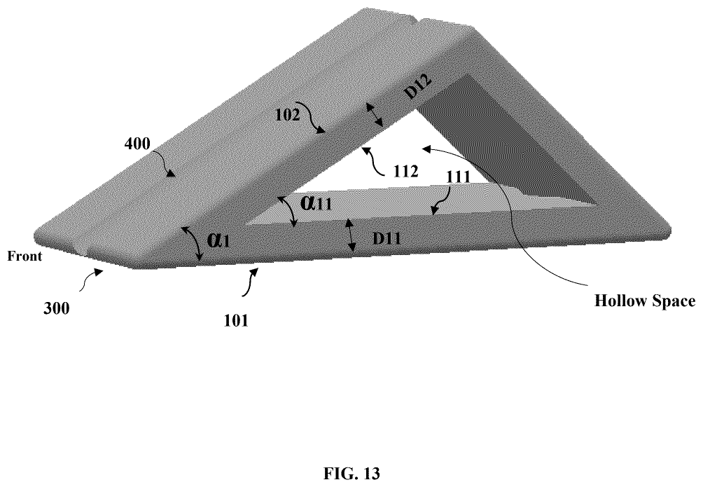

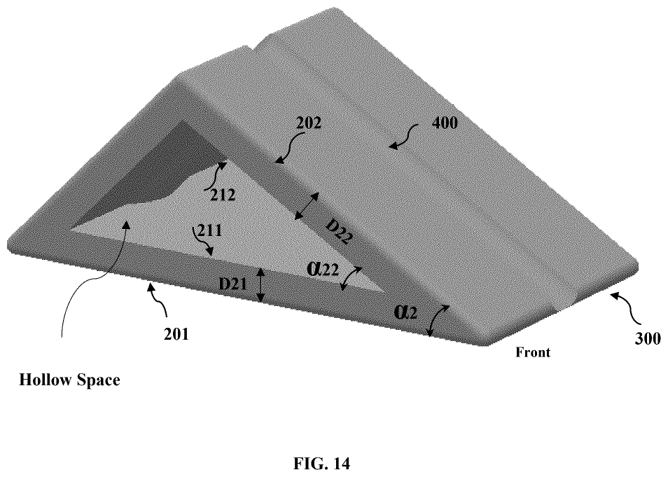

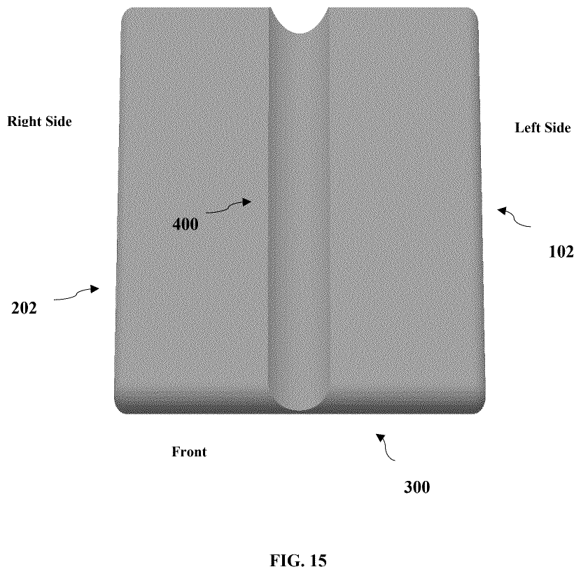

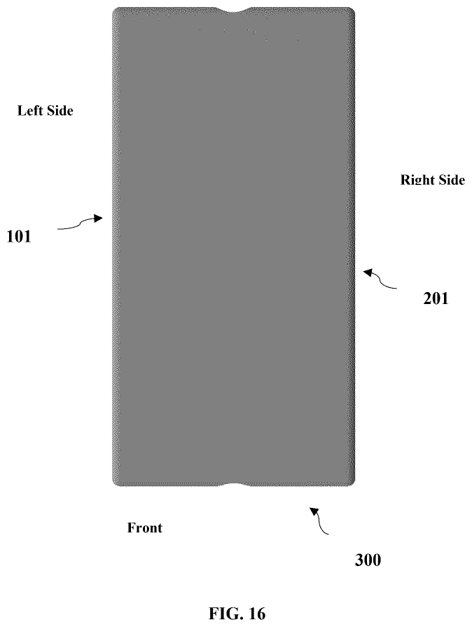

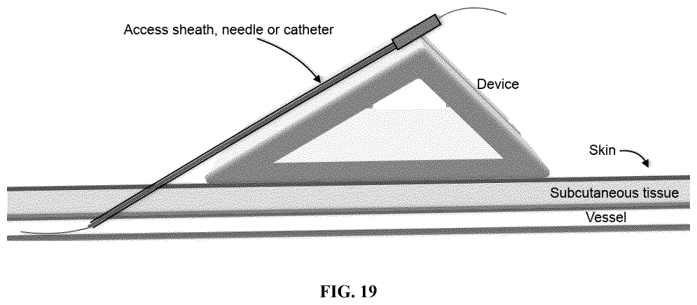

In a fourth embodiment, the medical device has a wedge shape (a second wedge shape device). (FIGS. 13-16). The second wedge shape device includes and incorporates all the structural components of the third embodiments, i.e., top surface T, bottom surface B, side surfaces, edges 101, 111, 102, 112, 201, 211, 202, 212, 300, and optional groove 400, the angles of α1, α11, α2, and α22, the diameter of catheter the wedge device can hold, the bottom surface material, the bottom surface adhesive, and tapering of the edges. Furthermore, all the structural components of the second wedge shape device, i.e., top surface T, bottom surface B, side surfaces, edges 101, 111, 102, 112, 201, 211, 202, 212, 300, and optional groove 400, the angles of α1, α11, α2, and α22, the diameter of catheter the wedge device can hold, the bottom surface material, the bottom surface adhesive, and tapering of the edges, fall within the same ranges of these structural components of the third embodiments. Furthermore, each of the structural components of the second wedge device and the third embodiment shares the same preferred ranges, respectively. An example of the application of the second wedge shape device is demonstrated in FIG. 19, where the second wedge shape device provides stable support to access sheath, needle, guide wire, or catheter in the femoral arterial or venous access procedures.

The current disclosure further provides a fifth embodiment, which also has a wedge shape (a third wedge shape medical device). The third wedge medical device is identical to the second wedge shape medical device except that the third wedge device does not have a complete hollow interior. More specifically, there is a separator in the hollow interior space of the third wedge device, completely or partially dividing the hollow interior into two cavities.

Although the present invention has been described with reference to specific embodiments, also shown in the appended drawings, it will be apparent to those skilled in the art that many variations and modifications can be done within the scope of the invention as described in the specification and defined with reference to the claims below.

What is claimed is:

1. A medical device for supporting a catheter during a vascular procedure, comprising:
a bottom surface in contact with patient's body surface;
a top surface supporting the catheter; and
wherein one end of the top surface and one end of the bottom surface are connected forming a connection area or connection line;
wherein the top surface forms an inclined plane or a ramp with the connection area/line facing an access site or a skin puncture point of the catheter; and
wherein the top surface optionally comprise a groove to stabilize and guide the catheter during the vascular procedure.

2. The medical device of claim 1, wherein the angle between the top surface and bottom surface on the left end is α1 and the angle between the top surface and bottom surface on the right end is α2.

3. The medical device of claim 2, wherein α1 and α2 are independently from about 15 degrees to about 90 degrees.

4. The medical device of claim 3, wherein α1 and α2 are independently selected from about 45 degrees, about 60 degrees, and about 90 degrees.

5. The medical device of claim 4, wherein α1 and α2 are equal.

6. The medical device of claim 3, wherein the top and bottom surfaces are further connected by a left surface, a right surface, or both.

7. The medical device of claim 3, wherein the groove, when present, can hold a catheter having a diameter from about 2 mm to about 20 mm.

8. The medical device of claim 3, wherein the bottom surface is made of a material, which changes its shape when the device is pressed against the patient's body surface and is able to conform to the shape of patient's body surface.

9. The medical device of claim 8, wherein the material has an apparent hardness/elasticity durometer measurement ranging from 10 to 100 using the Shore 00 index, or from 10 to 90 using the Shore A index, or from 10 to 90 using the Shore D index.

10. The medical device of claim 3, wherein the device has a wedge shape.

11. The medical device of claim 10, wherein α1 and α2 are independently selected from about 45 degrees, about 60 degrees, and about 90 degrees.

12. The medical device of claim 11, wherein α1 and α2 are equal.

13. The medical device of claim 10, wherein the top and bottom surfaces are further connected by a left surface, a right surface, or both.

14. The medical device of claim 10, wherein the groove, when present, can hold a catheter having a diameter from about 2 mm to about 30 mm.

15. The medical device of claim 10, wherein the bottom surface is made of a hard material, which does not change its shape when the device is pressed against the patient's body surface.

16. The medical device of claim 10, wherein the bottom surface comprise a material, which the device to change its shape when the device is pressed against the patient's body surface and is able to conform to the shape of patient's body surface.

17. The medical device of claim 16, wherein the material has an apparent hardness/elasticity durometer measurement ranging from about 10 to about 100 using the Shore 00 index, or from about 10 to about 90 using the Shore A index, or from about 10 to about 90 using the Shore D index.

18. The medical device of claim 10 may exhibit tapering on all edges where every two sides meet with a diameter ranging from 1 mm to 5 mm.

19. The medical device of claim 3, wherein the bottom surface comprises an adhesive allowing the medical device to be secured in position.

20. The medical device of claim 14, wherein the device comprises at least the top, bottom, left and right surfaces, and the interior of the device is hollow allowing a strap or bandage to be passed through it to secure the wedge in position on the body of the patient or bed.

21. The medical device of claim 20, wherein the hollow space has its own top and bottom surfaces and the angle between the top surface and bottom surface of hollow space on the left end is α11 and the angle between the top surface and bottom surface of the hollow space on the right end is α22.

22. The medical device of claim 21, wherein α11 and α22 are independently from about 15 degrees to about 90 degrees.

23. The medical device of claim 22, wherein α11 and α22 are independently selected from about 45 degrees, about 60 degrees, and about 90 degrees.

24. The medical device of claim 23, wherein α11 and @22 are equal.

25. The medical device of claim 24, wherein α1, α2, α11, and α22 are all equal.
